(19) United States
(12) Patent Application Publication
(10) Pub. No.: US 2007/0073224 A1
(43) Pub. Date: Mar. 29, 2007

(54) Syringe with latching safety mechanism

(76) Inventor: John Dries, Louisville, KY (US)

Correspondence Address: John Dries, 1509 Edgewood Place, Louisville, KY, 40205, US

(21) Appl. No.: 11/181,518

(22) Filed: Jul. 14, 2005

(51) Int. Cl. A61M 5/00

(52) U.S. Cl. 604/110

(57) ABSTRACT

A safety syringe having a safety shield (28) that can be repeatedly latched into the extended and retracted positions or permanently locked into the extended position. The syringe includes a barrel (34) that has gripping surfaces (76) that aid the user in controlling the syringe and naturally positions the user's hand for operating the plunger (24) and the safety shield's actuator (30). The syringe also includes a gear (52) and rack (56, 58) mechanism which moves the safety shield (28) a further distance than the user pushes the actuator (30).

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### FIELD OF THE INVENTION

The present invention relates to medical devices with safety sharps features. More specifically, the present invention relates to safety needle and syringe devices having self-contained safety shields that can be positioned to help prevent accidental needle sticks.

### BACKGROUND OF THE INVENTION

Many medical procedures require the use of metallic needle cannula for obtaining fluid samples from patients or for delivering fluids and medicines to the patient. Because sharper needles generally cause less pain to the patient when they are inserted into the skin, needle cannula for use in syringes and other medical devices have been developed which are extremely sharp and therefore cause less discomfort for the patient. While the sharpness of the needles is generally desirable for the patient they can also more easily cut or puncture the skin of the patient, doctor or nurse if the needle inadvertently touches the skin. These inadvertent “needle sticks” can pose a serious health hazard to anyone handling needles or in close proximity to needle or “sharps” devices. Because of the accidental nature of these needle sticks they can sometimes be relatively forceful and cause wounds which are subject to infection even if the needle is sterile. An accidental needle stick with a used needle cannula can transmit serious infections from blood born pathogens.

In order to make needles and syringes safer for patients and health care workers a variety of safety shields and devices have been developed for use with syringes and needles. There are four general types of safety devices currently in use:

(a) Safety devices which utilize a shield that telescopes over the sharp tip of the needle and then permanently locks into place preventing further access to the needle tip. Such a syringe is taught in U.S. Pat. No. 5,674,203 to Lewandowski. This syringe contains a cylindrical sheath that can be advanced axially to a locked irreversible position that prevents further access to the needle cannula tip. The cylindrical sheath sleeve is large enough in diameter to fit over the body of syringe when not in use and is pushed by the user into the extended and locked position. Once locked in place an indicator is provided which shows if the sheath is fully locked in place.

(b) Safety shields that use a pivoting shield that are rotated over the needle and permanently locked into place after the needle has been used. Such a syringe is taught in U.S. Pat. No. 6,780,169 to Crawford. This design uses a safety shield assembly having a shield and collar for connecting the shield to a syringe or fluid-handling device whereby the shield may be pivoted with respect to the collar. Once the device has been used, the user utilizes their hand or hard surface to rotate and lock the pivoting shield over the needle. Once locked into place the needle is no longer accessible and the device can be more safely disposed of

(c) Safety shields that slide along the length of the needle cannula and then cover and lock in place over the tip of the needle. Such a syringe is taught in U.S. Pat. No. 6,635,032 to Ward. This safety needle assembly uses a shield that is mounted for sliding movement along the needle cannula. The shield assembly also includes a plurality of arms articulated to one another to permit sliding movement along the needle cannula from its proximal position to its distal position. The arms are held during usage of the safety needle. Upon release of the arms, a spring automatically drives the shield to the distal position. Once in the distal or fully extended position the tip of the needle is covered and the shield locks into place.

(d) Safety devices that utilize retracting needles. Such a syringe is taught in U.S. Pat. No. 6,010,486 to Carter. This retracting needle syringe uses a spring loaded needle which retracts inside of the syringe once the syringe plunger is fully pressed into the barrel. Once the plunger is at the end of its travel, the user increases the axial force on the plunger in order release the needle and allow it to retract into the hollow interior of plunger. Once retracted the needle cannula tip is no longer accessible and the syringe can be more safely disposed of.

Each of these four basic types of safety systems has its own disadvantages. They also share the following common disadvantages.

The telescoping, sliding and rotating type shields require the user to either reposition the syringe in their hand or use both hands in order to move the shied into place over the needle cannula. During use of the syringe, repositioning the syringe in one hand may be awkward and both hands may not be available especially if the patient faints, pulls back or reacts unpredictably in response to the procedure. Often the healthcare worker needs to use their free hand to help support the patient's limb. With these types of devices it is also possible for the hand that is attempting to activate the safety shield to slip past the shield and come in contact with the sharp end of the needle cannula, thus increasing the possibility of an accidental needle stick.

Some manufactures of these devices recommend using a nearby hard, flat surface in order to help activate the shield and prevent the hand from having to come closer to the needle tip. The problem with this approach is that the contaminated needle needs to be transported to a hard surface with the contaminated needle exposed to everyone in the vicinity. Once at the hard surface, such as a tabletop, blood borne pathogens from the used needle cannula can contaminate the surface.

These types of safety devices also become more bulky and awkward to use as the length of needle cannula used becomes longer. If a longer needle is used, the telescoping, sliding or rotating guard must also be made longer in order to reach and cover the tip of the needle.

The type of safety syringes which use a retracting needle also have disadvantages peculiar to that type of design. Because the area of the needle assembly where the needle cannula attaches to the syringe body is often made up of multiple small components, the rotational stiffness of the needle to the syringe body is lessened. Because of this, the needle cannula can rotate or deflect laterally when a side force is applied such as when the needle tip is pressed against the skin at an angle. This lateral deflection makes the needle more difficult to precisely control, especially if a relatively large amount of force is required to penetrate to the desired location below the skin. Lateral deflection also makes cleanly finding and inserting into veins more difficult. Another problem with the retractable type syringes is that the plunger must be fully depressed until the end of its travel is reached before the needle can be retracted. This makes this type of syringe inappropriate for drawing blood or aspiration procedures. Also, if the health care worker does not wish to inject the full volume of fluid contained in the syringe, the needle cannot be retracted until all of the contents of the syringe have been expelled into some sort of container. The fluid which must be expelled before the needle can be retracted must pass through the used cannula and can therefore become contaminated with blood borne pathogens.

All four types of safety devices discussed above suffer from the following problems:

(a) The fact that all of these safety systems can only be engaged once means that any transportation, manipulating or holding of the device while waiting to be used is done with an unprotected needle exposed to everyone in the vicinity. Commonly, a safety cap that is shipped with the needle or syringe is removed before the needle tip is inserted into a conventional container with medicine or fluid and then drawn or aspirated into the syringe. From the moment the shipping safety cap is taken off of the syringe until after the syringe has been used and the health care worker gets in a position to activate the safety device the needle cannula is exposed and poses a sharps hazard. If the uncovered needle needs to be transported any distance before use, the separate needle cover can be put back over the needle cannula. Reinstalling the needle cover that came with the new needle has proven to be an awkward and hazardous procedure. If the healthcare worker slightly misjudges the alignment or is disturbed during the procedure there is chance that the healthcare worker will be stuck by the needle.

(b) Because most of these syringes depend on very simple syringe barrel designs, the location where the healthcare worker grips the syringe body with their fingers is usually a cylinder with a flat flange at the top. This type of gripping surface provides good axial stability in only one direction and little lateral or rotational stability. Because of this, conventional syringes can be difficult to control with one hand when precision is required in the injection or aspiration procedure.

(c) It is often necessary to use a syringe more than one time in certain procedures such as knee aspirations. With the current type of single use shields the needle is exposed and is a sharps hazard to everyone in the vicinity until the procedure is completed and the syringe can be permanently disposed of

### BACKGROUND OF INVENTION—OBJECTS AND ADVANTAGES

Accordingly, several objects and advantages of the present invention are:

(a) to provide a safety shield which can be repeatedly engaged and disengaged allowing the safe transport and use of the syringe after it has been charged with fluid or while being used in medical procedures.

(b) to provide a safety shield which can be easily engaged and disengaged with one hand without requiring that the user move or reposition his or her hand.

(c) to provide a safety shield which can automatically eject the shipping safety cap without forcing the user to put his or her hand near the sharp needle cannula.

(d) to provide a syringe barrel which offers the user a gripping surface that provides improved axial, lateral and rotational stability, precision and control.

(e) to provide a shield and shield actuation means which do not become excessively bulky or difficult to use as needle length increases.

Further objects and advantages will become apparent from a consideration of the drawings and ensuing description.

### SUMMARY OF THE INVENTION

A safety needle device of the present invention includes a barrel having an inside surface defining a chamber and an open proximal end. The barrel includes a distal end having a needle cannula attached thereto. The needle cannula is in fluid communication with the chamber. The needle cannula projects distally outwardly from the distal end of the barrel and terminates in a distal tip. A safety shield is mounted on the barrel for movement relative to the barrel from a retracted position in which the safety shield does not materially obstruct access to the distal tip of the needle cannula and an extended position in which the safety shield prevents access to the distal tip. The safety needle device includes an actuator means for securely and repeatedly retaining the needle guard in the retracted position, extending and latching the guard in the extended position, then unlatching and retracting the shield back into the retracted position. A gear is mounted to the actuator that is allowed to rotate relative to the actuator but is restrained to move axially with the actuator. The gear's teeth mesh with rack teeth molded into the syringe barrel and with rack teeth molded into the safety shield. Pushing down on the actuator causes the actuator and gear to move relative to syringe barrel, the gear teeth meshing with the rack teeth molded into the barrel causing the gear to rotate as well as translate. The translating and rotating gear meshes with the rack teeth molded into the safety shield thus causing the safety shield to translate twice the distance that the actuator travels. After the shield has been extended and retracted as many times as required, the shield can be permanently locked into the extended position by pushing the actuator past its normal travel until a one-way snap fit is reached. The syringe includes a shipping safety cap that can be ejected by the shield as it moves from the retracted to the extended position. The syringe barrel has grooves molded into the exterior surface that serve the dual function of retaining and guiding the actuator and safety shield as well as providing a superior gripping surface for the user's fingers to grip and control the syringe barrel during use.

### BRIEF DESCRIPTION OF THE DRAWINGS—FIGURES

FIG. 1 is a perspective view of the syringe assembly with the plunger extended and the shipping safety cap installed over the top of the needle.

FIG. 2 is another perspective view of the syringe assembly with the plunger extended and the shipping safety cap removed.

FIG. 3 is a side view of the syringe assembly with the plunger fully depressed and the shipping safety cap removed.

FIG. 4 is a perspective view of the syringe assembly with the plunger fully depressed and the safety shield latched in the extended position.

FIG. 5 is a side view of the syringe assembly with the plunger fully depressed and the safety shield latched in the extended position.

FIG. 6 is a top view of the syringe assembly with the plunger fully depressed and the safety shield latched in the extended position.

FIG. 7 is a perspective view of the unassembled or “exploded” pieces of FIG. 1.

FIG. 8 is a perspective view of the barrel.

FIG. 9 is another perspective view of the barrel.

FIG. 10 is a side view of the barrel.

FIG. 11 is a perspective view of the latch.

FIG. 12 is a perspective view of the thrust link.

FIG. 13 is a perspective view of the gear.

FIG. 14 is a perspective view of the safety shield.

FIG. 15 is a perspective view of the actuator with its hinged cap shown in the fully open position.

FIG. 16 is a perspective view of the actuator with its hinged cap in the fully open position and the latch, gears and thrust links shown assembled in place.

FIG. 17 is a perspective view of the actuator with its hinged cap in the closed position and the latch, gears and thrust links shown assembled in place.

FIG. 18 is a close-up perspective view of the latch in place on the actuator.

FIG. 19 is a top view of the syringe assembly with the shipping safety cap, spring and plunger removed.

FIG. 20A is a cross sectional view of the syringe assembly as shown in FIG. 19 along line 20-20 thereof showing the latching mechanism for the safety syringe in the retracted position of the device.

FIG. 20B to 20F are cross sectional views of the syringe assembly as shown in FIG. 19 along line 20-20 thereof showing the operation of the latching mechanism in transferring the safety shield from the retracted position to the extended and latched position.

FIG. 20G to 20I are cross sectional views of the syringe assembly as shown in FIG. 19 along line 20-20 thereof showing the operation of the latching mechanism transferring the safety shield form the extended and latched position to the retracted position

FIG. 20J is a cross sectional view of the syringe assembly as shown in FIG. 19 along line 20-20 thereof showing actuator locked into the extended position.

FIG. 21 is a perspective view of the actuator.

### DETAILED DESCRIPTION—PREFERRED EMBODIMENT

While this invention is satisfied by embodiments in many different forms, there is shown in the drawings and will herein be described in detail, the preferred embodiments of the invention, with the understanding that the present disclosure is to be considered as exemplary of the principles of the invention and is not intended to limit the invention to the embodiments illustrated. Various other modifications will be apparent to and readily made by those skilled in the art without departing from the scope and spirit of the invention. The scope of the invention will be measured by the appended claims and their equivalents.

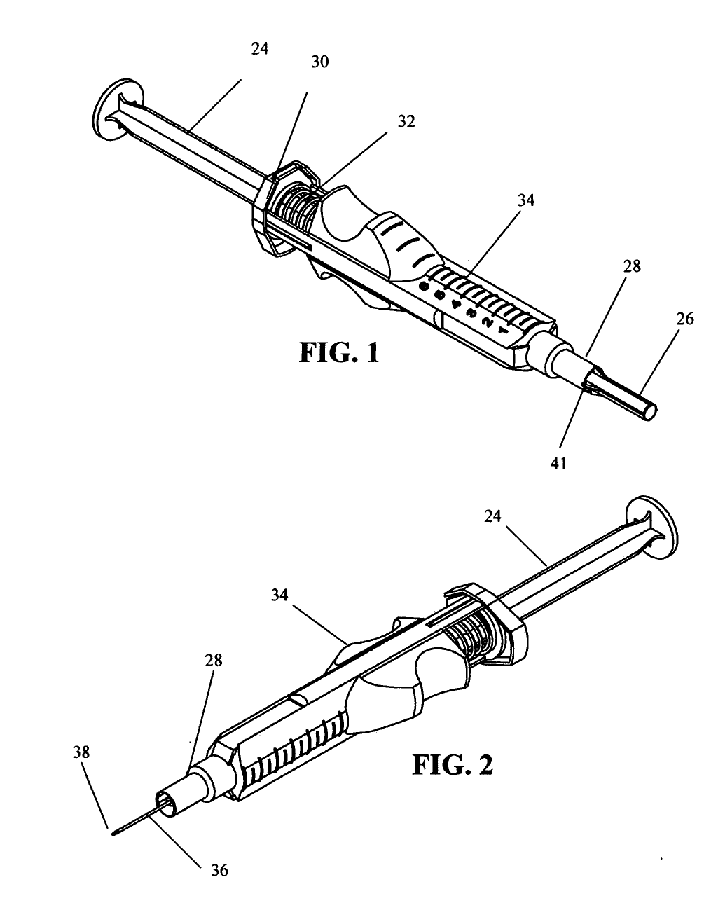

Referring to the drawings in which like reference characters refer to like parts throughout several views thereof, FIG. 1 illustrates a syringe assembly with the safety mechanism of the present invention in the retracted position and a plunger 24, in the extended position. The syringe assembly includes a shipping safety cap 26, a safety shield 28, an actuator 30, a return spring 32, and a barrel 34.

For the purposes of the description of the present invention, the term “distal end” is intended to refer to the end of the syringe assembly furthest from the person holding the syringe, whereas the term “proximal end” is intended to refer to the end closest to the holder of the syringe.

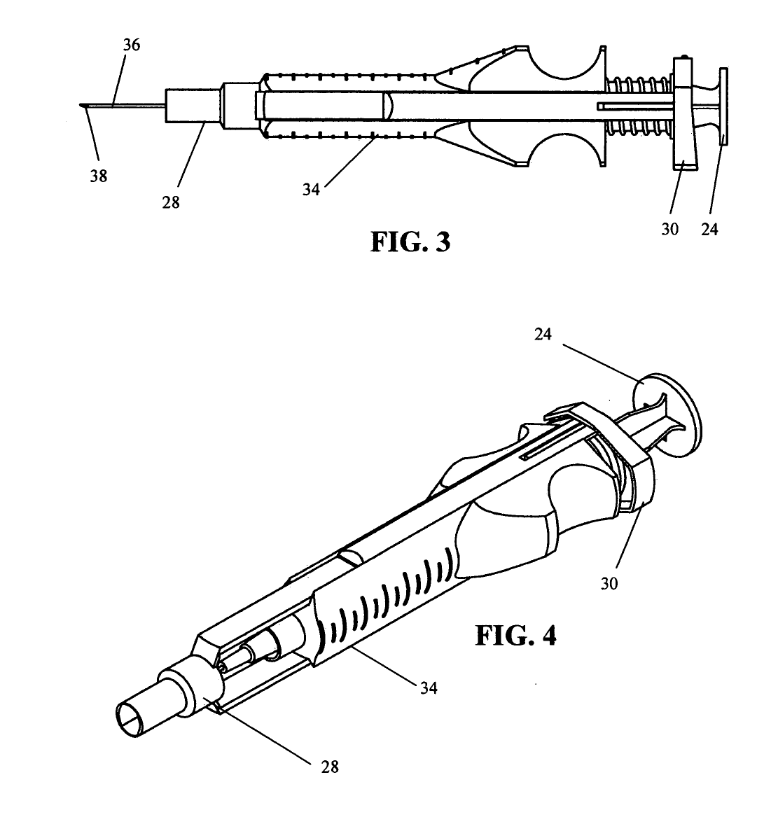

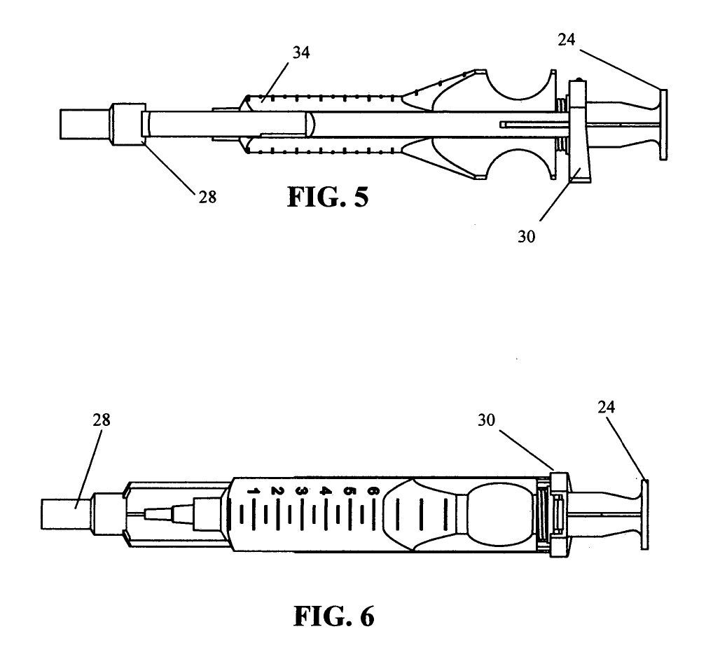

As shown in FIGS. 2 and 3, the cap 26 has been removed from the syringe assembly revealing a needle cannula 36. The safety shield 28 fits over the needle 36 and is mounted on the barrel 34 for movement relative to the barrel from a retracted position in which the needle guard does not materially obstruct access to distal tip 38 of the needle cannula 36 and in the extended position, as shown in FIGS. 4-6, in which the safety shield 28 obstructs access to the distal tip 38 of the needle cannula 36.

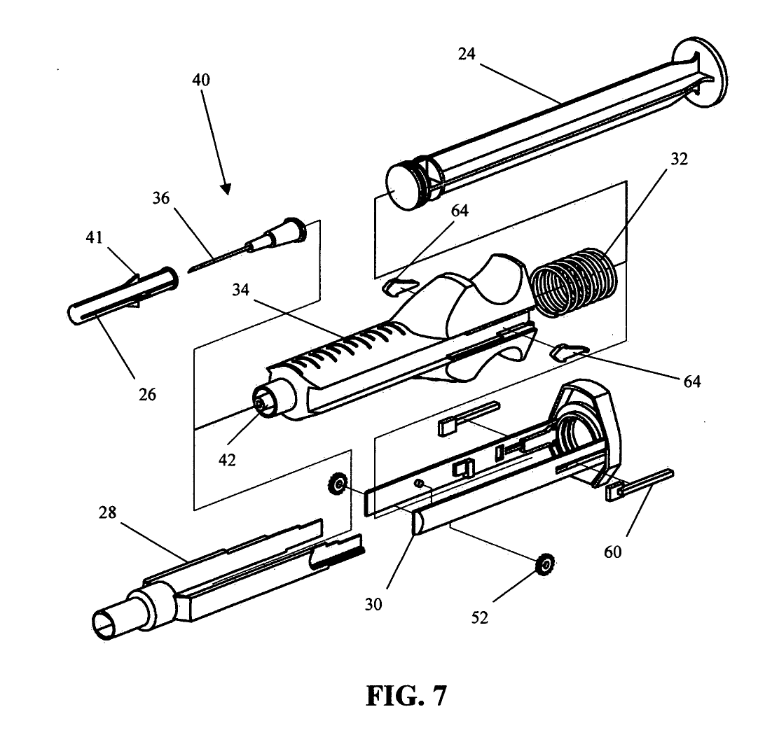

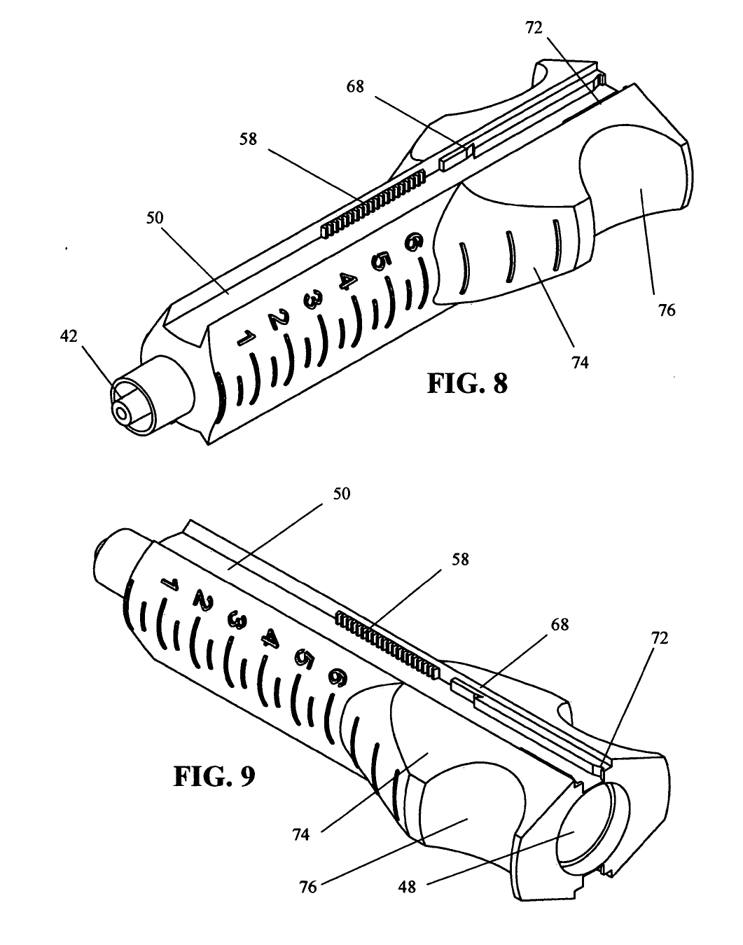

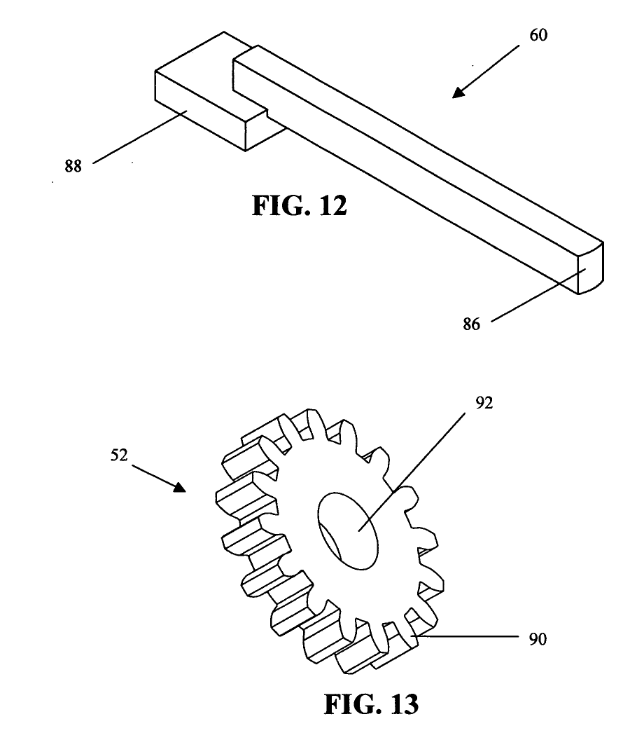

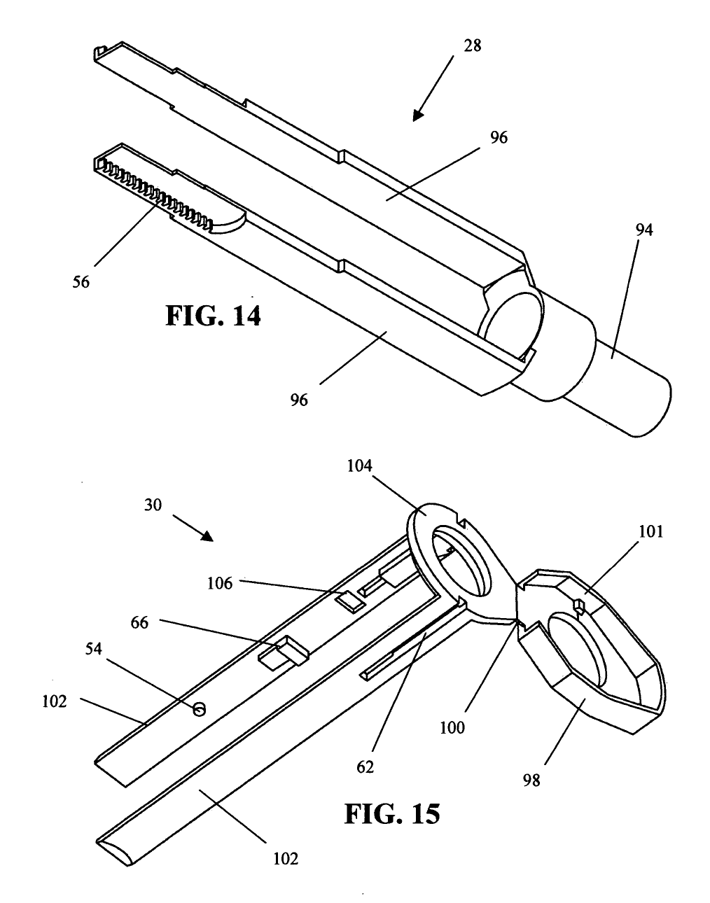

As shown in FIGS. 7, 9, 14, 16 a needle assembly 40, mounts onto the distal end 42, of the barrel 34 (FIG. 7). The return spring 32 fits against the proximal end of the barrel 34 (FIGS. 7, 9). The plunger 24 is inserted through the spring 32 and into the cylindrical interior 48 of the barrel 34 (FIG. 7). The cylindrical interior of the barrel 48 is shown in FIG. 9. The shield 28 mounts into guide grooves 50 which are molded into the sides of the barrel 34 (FIGS. 7-9). The shipping safety cap 26 is mounted over the top of needle assembly 40 (FIG. 7). Ejection tabs 41 project radially from the shipping safety cap 26. A gear 52, mounts onto an axle 54 molded into each side of the actuator 30 (FIGS. 7, 15-17). When fully assembled, the gears 52 mesh with rack teeth 56 formed into the safety shield 28 (FIG. 14) and rack teeth 58 molded into the barrel 34 as shown in FIGS. 8 and 9. A thrust link 60 fits into slots 62 formed into each side of the actuator 30 (FIGS. 12, 15). A latch 64 fits into grooves 50 molded into each side of the barrel 34 (FIGS. 7, 9). When assembled, the latch 64 is trapped between the walls of the groove 50, the thrust link 60 on the proximal of the latch 64 and a projected drive surface 66 on the distal end of the latch 64.

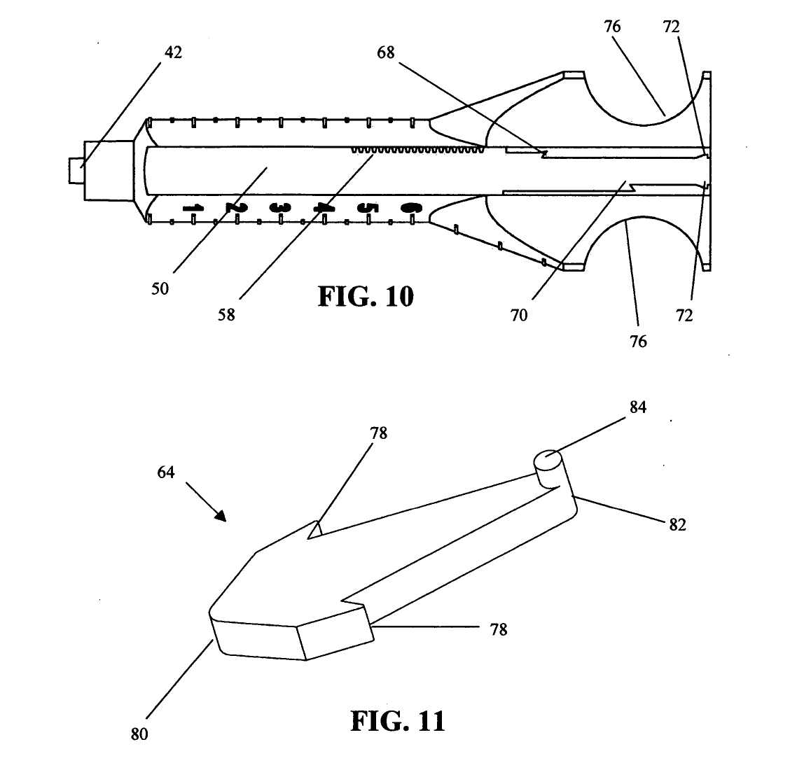

As shown in FIGS. 8-10, the barrel 34 has guide grooves 50 formed into the sides of the barrel 32. Along the length of the guide grooves 50 are rack teeth 58 molded into one side of each guide groove 50. Near the proximal end of the barrel 50 is an extended latch means 68, and nearer to the proximal end of the barrel is a retracted latch means 70. A locking notch 72 is molded into each side of the guide grooves 50. The raised material 74 on the outside of the barrel 32 used to form the guide grooves 50 of sufficient depth to completely retain the shield 28 and actuator 30 also provide the required material to form a gripping surface 76.

As shown in FIG. 11, the latch 64 includes latch arms 78 on each side. A fulcrum 80 is located at the base of the latch 64 and a drive surface 82 is located at the top of latch 64. At the top of latch 64 is a guide post 84 which also forms a portion of the drive surface 82.

As shown in FIG. 12, the thrust link 60 has a drive surface 86 at the top and a pushing block 88 at its base.

As shown in FIG. 13, the gear 52 has teeth 90 around its perimeter and a cylindrical opening 92 in its center.

As shown in FIG. 14, the safety shield 28 has a cylindrical needle shield 94 at its distal end. Guide arms 96 extend from each side of the needle shield 94. Rack teeth 56 are molded into the proximal ends of the guide arms 96.

As shown in FIG. 15, the actuator 30 has a pivoting cap 98 which is connected to the rest of the actuator 30 by a “living hinge” 100, that is a thin flexible strip of plastic connecting the cap 98 to the rest of the actuator 30. The underside of the actuator cap as shown in FIG. 15 has thrust projections 101 which align with the slots 62 when the actuator cap 98 is in its closed position as shown in FIG. 17. Guide arms 102 extend from the proximal end 104 of the actuator 30. An axle 54 projects from the inside surface of each guide arm 102. A projected drive surface 66 projects from the inside surface of each guide arm 102. A guide post slot 106 is molded into the inside surface of each guide arm 102. A slot 62 is molded into each guide arm 102 and continues up to and through the proximal end 104 of the actuator 30. A thumb depression 110 is molded into the top of the cap 98 as shown in FIG. 17.

As shown in FIG. 21, a pair of locking tabs 108 are located on the inside surface of each guide arm 102.

### DETAILED DESCRIPTION—OPERATION

The safety syringe of the present invention is operated in the following manner: The user grips the syringe barrel 32 in a conventional manner, which is gripping the proximal end of the barrel with their index and middle fingers. The index and middle fingers naturally tend to align with the gripping surfaces 76 (FIG. 8-10). When gripping the syringe in this manner, the user's thumb will naturally be in convenient alignment with the thumb depression 110 molded into the top of the actuator cap 98 (FIG. 17). When the user is ready to make use of the safety syringe, the user presses against the depression 110 molded into the actuator cap 98 with their thumb, which causes the safety shield 28 to begin to move from the retracted position toward the extended position. As the safety shield 28 moves toward the extended position, the safety shield pushes against the ejection tabs 41 on the shipping safety cap 26 (FIG. 1). As the safety shield 28 moves from the retracted position to the extended position, the shipping safety cap 26 moves with it. When the shield 28 is fully in the extended position (FIGS. 4-6) it latches in place and cannot be retracted until the actuator cap 98 is again pressed, unlatching the shield 28 and allowing the return spring 32 to move the shield 28 back to the retracted position. With the safety shield 28 latched in the extended position the shipping safety cap 26 is no longer engaged with the needle assembly 40 and can be placed or allowed to fall into a waste container. Because the safety shield 28 simultaneously prevents access to the distal tip 38 of the needle cannula 36 and ejects the shipping safety cap 98 the user is never exposed to the sharp distal tip of the needle 36 while ejecting the safety cap 26.

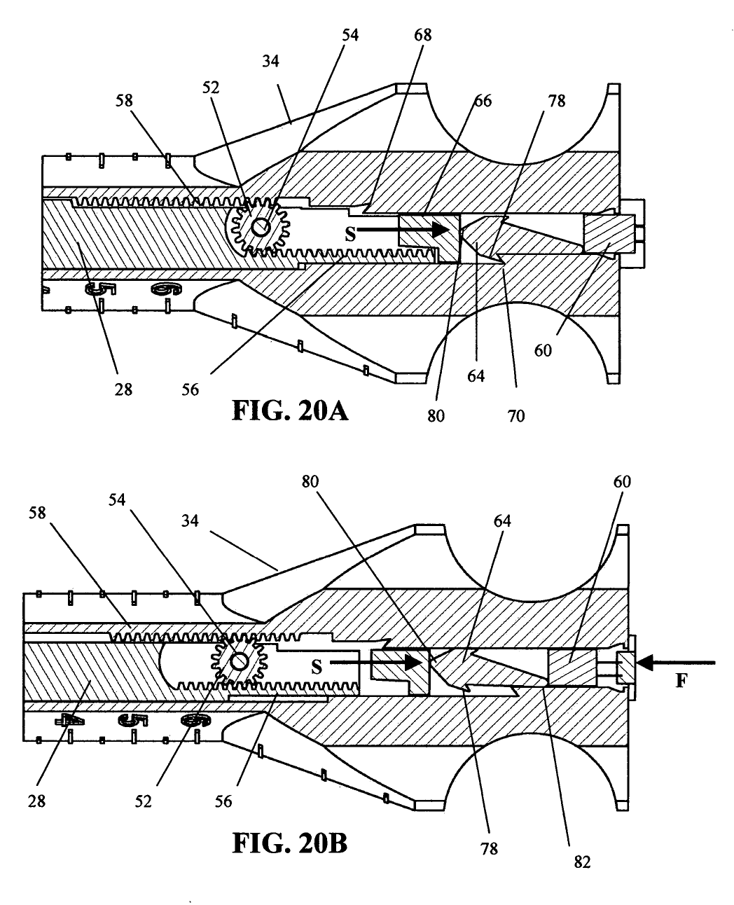

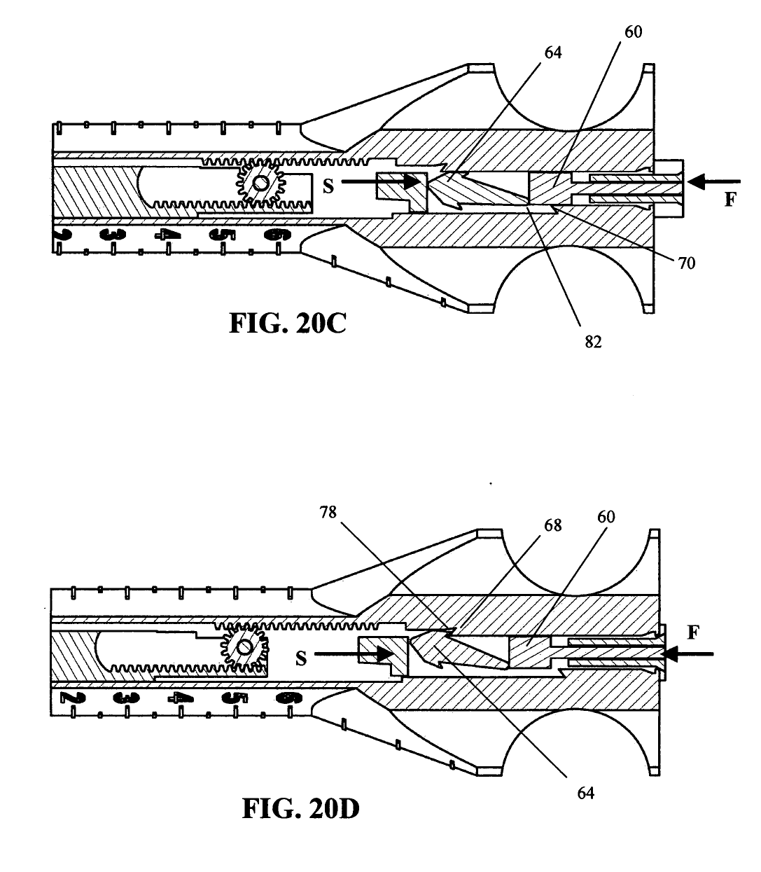

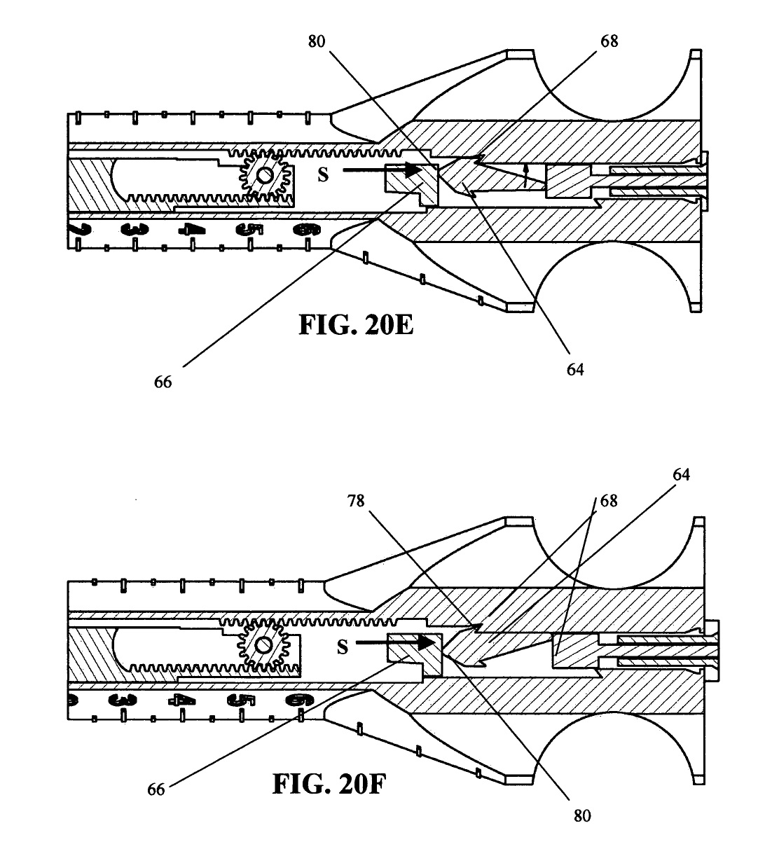

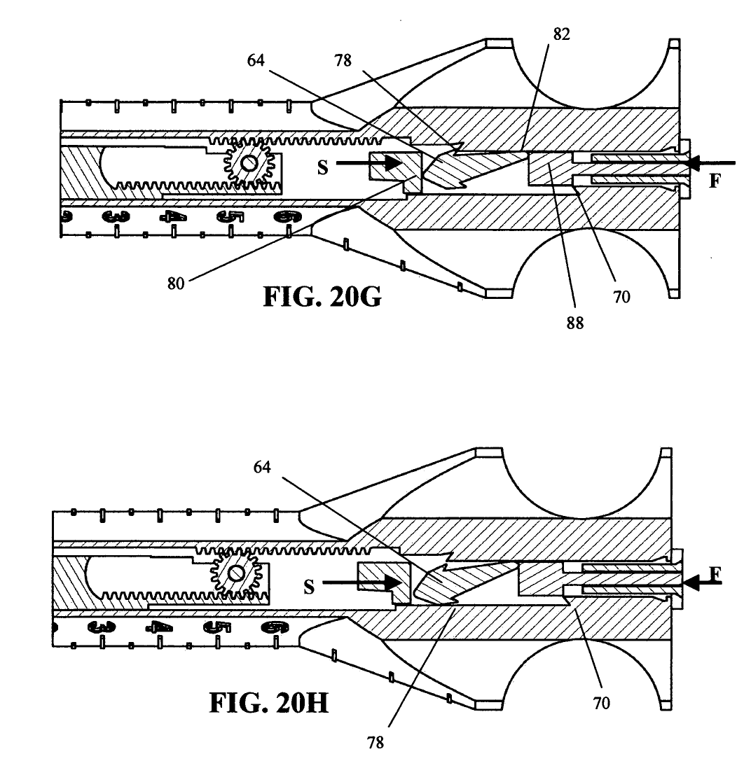

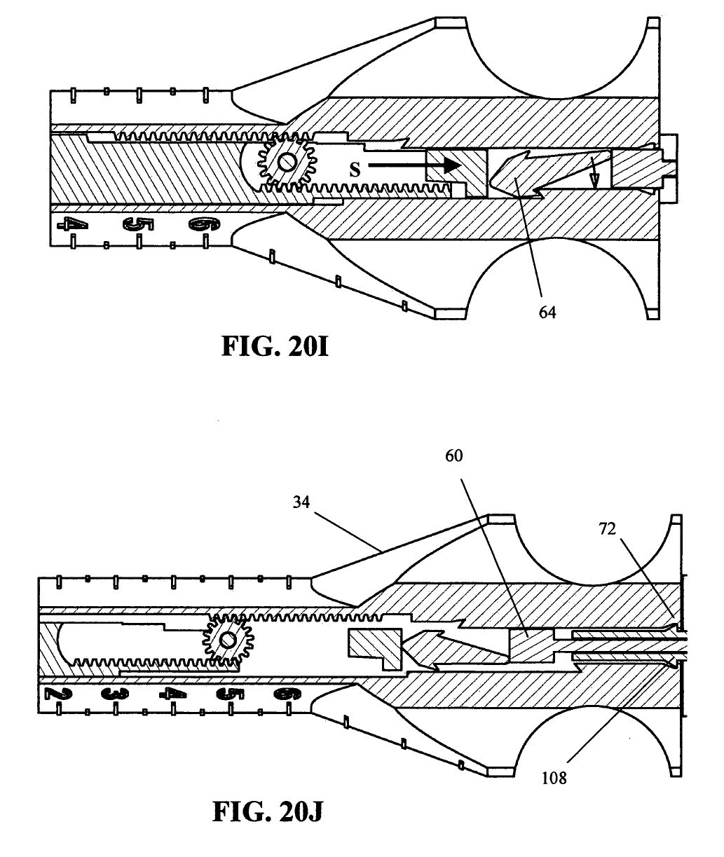

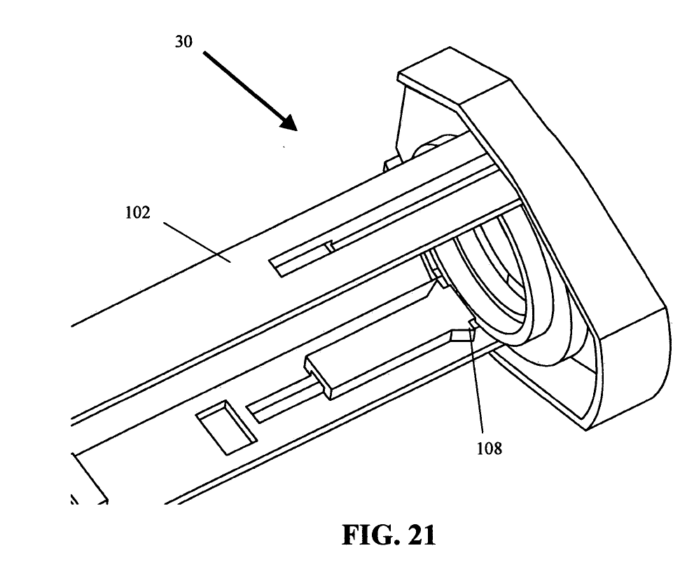

Once the shipping cap 26 has been ejected and the safety shield 28 is latched in the extended position, the syringe can safely be transported within the workspace the healthcare worker is operating. Often at this point in a syringe's use medicine or fluid is drawn into the syringe in order to be injected into the patient. Often this fluid or medicine is stored in conventional container (not shown) with a membrane covering the top of the container. The membrane is usually designed to be easily penetrated by a needle cannula so that medicine can then be drawn into the syringe through the membrane by pulling the plunger 24 in the proximal direction. When the syringe is filled with the desired amount of fluid or medicine, the needle cannula is withdrawn from the container. With the present invention when the user has the conventional medicine container in place and is ready to press the needle cannula 36 into the permeable membrane of the medicine container the safety shield 28 can then be easily released from the extended position and allowed to return to the retracted position by once again pressing the actuator cap 98 which in turn unlatches the safety shield 28 allowing the return spring 32 to return the shield 28 to the retracted position. Once the syringe is filled with the desired amount of fluid or medicine the safety shield 28 is once again latched into the extended position by pushing the actuator cap 98 until the mechanism once again latches in the extended position. With the safety shield 28 latched into the extended position the syringe assembly may once again be safely transported within the workspace. When the syringe is ready and in position to make an injection or aspiration procedure on the patient the safety shield 28 is once again set in the retracted position by pressing the actuator cap 98 thus unlatching the safety shield 28. When the injection or aspiration is complete the user once again latches the safety shield 28 into the extended position by pressing the actuator cap 98. Once the syringe is again latched into the extended position the syringe can be safely transported within the workspace or transported longer distances such as to a separate laboratory. If the user is completely finished with the syringe the actuator cap 98 can be pressed beyond its normal travel and permanently locked into the extended position (FIG. 20J). With the syringe's safety shield 28 permanently locked into the extended position the syringe cannot be reused and can be disposed of. It is important to note that each time the safety shield 28 is moved from the extended or retracted position the user does not have to use a second hand or reposition the hand which is operating the syringe.

The detailed function of each component of this preferred embodiment is as follows:

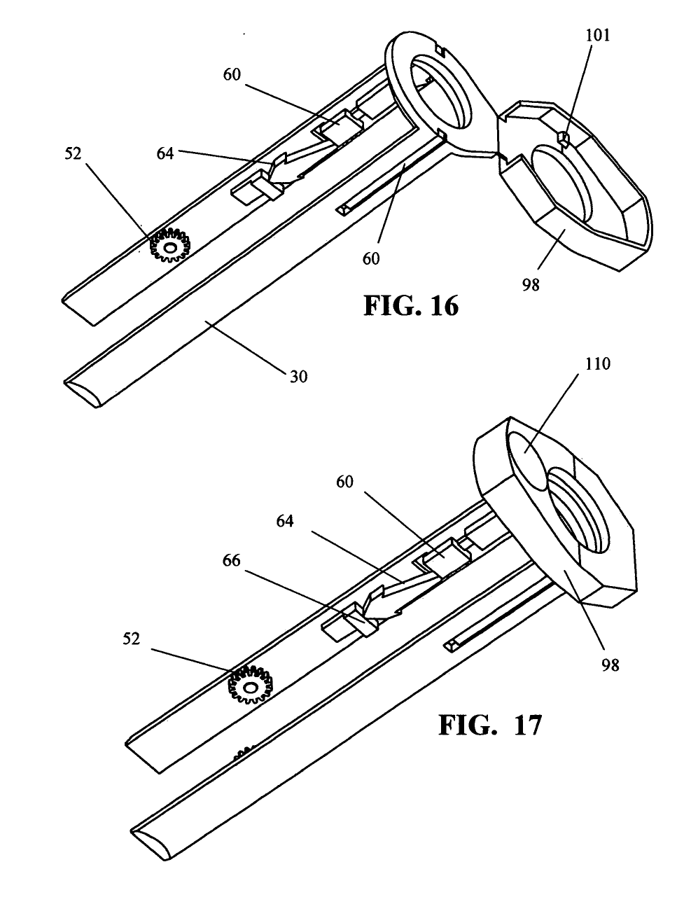

FIG. 16 shows the actuator 30 with the actuator cap 98 in the open position. The gear 52, latch 64 and thrust link 60 are shown assembled into place relative to the actuator 30. When the cap 98 is closed and the user presses against the thumb depression 110 molded into the top of the actuator cap 98 (FIG. 17) the thrust projections 101 press against the drive surface 86 of the thrust link 60. The pushing block 88 of the thrust link 60 in turn exerts pressure on the drive surface 82 of the latch 64. The fulcrum 80 of the latch 64 in turn presses against the projected drive surface 66 of the actuator 30. In effect, when the thumb depression 110 is pressed by the user the force is transferred to the projected drive surface 66 which moves the entire actuator assembly in the distal direction. The distal motion of the actuator 30 is resisted by the return spring 32 which always provides a force that attempts to move the actuator 30 in the proximal direction and back to the retracted position.

As shown in FIG. 20B, when the thrust link 60 is pushed with enough force F to overcome the return spring force S the actuator 30 will begin to translate in the distal direction. When the actuator 30 moves relative to the barrel 34 the axle projections 54 also move relative to the barrel 34. The axle projections 54 pass through the cylindrical openings 92 in the gears 52. The teeth 90 of gear 52 mesh with the rack teeth 58 molded into the barrel 34. When the axle 54 translates relative to the barrel 34 the gear 52 translates also. Because the gear's teeth 90 are meshed with the stationary rack teeth 58 the gear 52 is driven to rotate as well as translating with the actuator 30. The gear 54 also meshes the rack teeth 56 of the safety shield 28. The translating and rotational motion of the gear 54 drives the safety shield 28 twice the distance that the actuator 30 moves. This multiplication of linear motion allows the user to employ a smaller, quicker stroke to engage and disengage the safety shield 28. The overlapping nature of the guide arms 102 of the actuator 30 with the guide arms 96 of the safety shield 28 (FIGS. 7, 14, 15) allow syringes using relatively long needles to have a shield which does not become appreciably larger and bulkier as the amount of extension required to cover the needle is increased.

The operation of the latching mechanism is shown in FIGS. 20A-20I. The latch arms 78 of the latch 64 and their associated extended latch means 68 and retracted latch means 70 have co-operating means to facilitate a pivot action therebetween, and wherein when, following the depression of the actuator cap 98 to release the last engaged latch arm 78.

FIG. 20A shows the latch mechanism in the retracted state with one a latch arm 78 engaged with the retracted latch means 70. Due to the force S exerted by the return spring 32 on the actuator 30 the projected drive surface 66 pushes the fulcrum 80 of the latch 64 in the proximal direction. The line of action of the forces created by the drive surface 66 and the latch arm 78 engaged with the retracted latch means 70 create a pivot action which tends to hold the latch 64 in place.

FIG. 20B shows a force F from a user pressing on the actuator cap 98 which in turn pushes the thrust link 60 which in turn pushes the latch 64 in the distal direction. The line of action of forces acting on the fulcrum 80 and the drive surface 82 of the latch 64 maintains the pivoting action causing the opposite latch arm 78 to be pressed and slide against the alternate side of the groove 50 in the barrel 34.

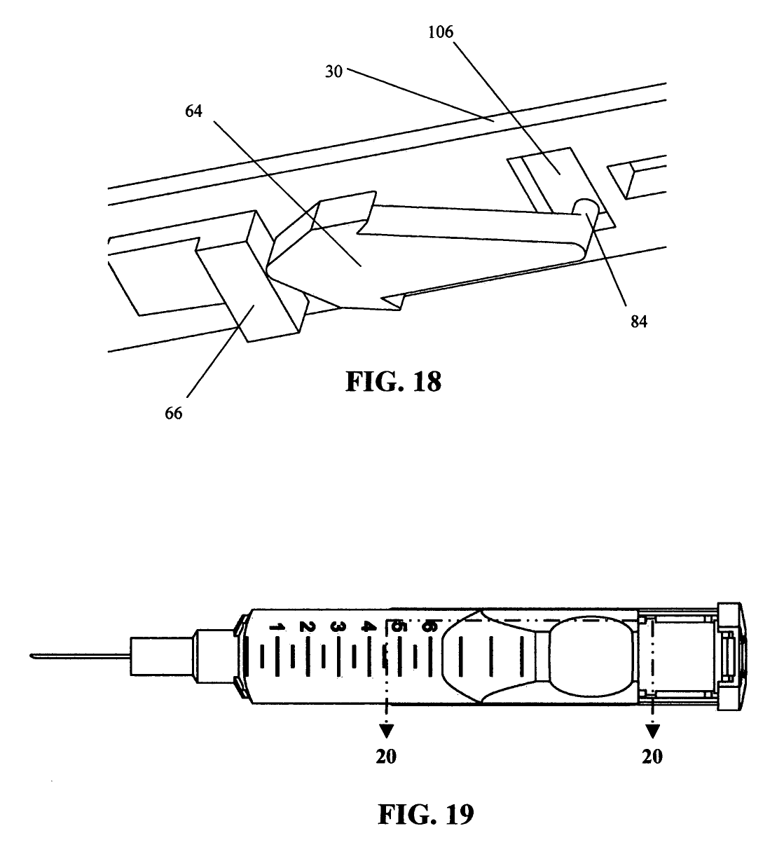

FIG. 20C shows thrust link 60 pressed further in the distal direction where the drive surface 82 of latch 62 has traveled past the retracted latch means 70. In order to prevent the drive surface 82 of the latch 62 from accidentally engaging with the retracted latch means 70 when the actuator is traveling back to the retracted position from the extended position a guide post 84 (FIG. 11) is incorporated into the latch 62. The guide post 84 fits into a guide post slot 106 which is molded into the actuator 30 as shown in FIG. 18. The guide post guide slot 106 prevents the drive surface 82 of the latch 64 from accidentally engaging with the retracted latch means 70. The guide post 84 and the mating guide post slot 106 allow the latch mechanism to work reliably with relatively long distances between the retracted and extended positions.

FIG. 20D shows the thrust link 60 pushed to just beyond the extended position. When in this position the latch 64 is allowed to further pivot where the latch arm 78 can engage the extended latch means 68.

FIG. 20E depicts the force F from the user no longer being applied and the line of action from the force S from the return spring 32 acting through the projected drive surface 66 to the fulcrum 80 of the latch 64 causing a pivoting action in the opposite direction of latch 64.

FIG. 20F depicts the force S of the return spring 32 acting through the projected drive surface 66 on the fulcrum 80 holding the latch 64 in place against the extended latch means 68 and the wall of the groove 50 (FIGS. 8-10).

FIG. 20G shows force F from the user moving the latch 64 in the distal direction. The pivoting action from the couple of the forces from the projected drive surface 66 acting on the fulcrum 80 and the pushing block 88 acting on the drive surface 82 of the latch 64 begin to rotate latch 64 so that the latch arm 78 will be in alignment to engage the retracted latch means 70.

FIG. 20H shows the latch 64 rotated so that latch arm 78 is sliding against the wall of groove 50 (FIGS. 8-10) so that the latch arm 78 is in alignment with the retracted latch means 70.

FIG. 20I shows the latch 64 rotating into position so that it will be held in place against the retracted latch means 70 by the force S of the return spring 32.

FIG. 20J shows the thrust link 60 pushed beyond its normal travel allowing the locking tabs 108 (FIG. 21) on the actuator 30 to engage with the locking means 72 on the barrel 34. Once in this position the actuator 30 is permanently locked in the extended position.

### DETAILED DESCRIPTION—CONCLUSION, RAMIFICATIONS, AND SCOPE

Thus the reader will see that the syringe of the present invention provides a reliable, compact, easy to precisely control, highly versatile and simple to operate safety syringe. More specifically, the safety syringe of the present invention has advantages that:

(a) it permits the safety shield to be extended, latched into place and retracted again as many times as is required by the healthcare worker.

(b) because of the overlapping nature of the shield 28 with the actuator 30 it can be manufactured for use with longer needles without requiring that the safety mechanism become significantly longer or bulkier.

(c) it allows the user to use a relatively short motion of their hand or finger to fully cover longer needles with a safety shield.

(d) it allows the user to keep their hand in the primary use position when extending, retracting or permanently locking the shield 28 into place.

(e) it automatically ejects the shipping safety cap 26 thereby sparing the user from having to use their hand to do this potentially hazardous action.

(f) it can be gripped more easily and controlled more precisely.

Although the description above contains many specificities, these should not be construed as limiting the scope of the invention but as merely providing illustrations of some of the presently preferred embodiments of the invention. For example, the actuator 30 and shield 28 can be constructed with only one guide arm each. It could also be constructed to operate with only one latch 64, gear 52, and thrust link 60. The position that the user pushes the actuator 30 to latch and unlatch the shield can be in many places such as the side of the syringe instead of at its proximal end. The type and location of the spring 32 can also be changed to at or near the midsection of the syringe or at the distal end of the syringe for instance; the barrel 34 can be made with only one groove or many grooves. The shield and actuator assembly can be manufactured as part of the needle assembly. The gear ratios of between the gear, actuator and safety shield can be changed thus changing the amount of extension of the shield relative to the actuator. Also, the safety shield can be connected directly to the actuator, thus making the actuator and shield one piece completely eliminating the gear, etc.

Thus the scope of the invention should be determined by the appended claims and their legal equivalents, rather than by the examples given.

What is claimed is:

1. A safety needle device comprising:
a barrel having an inside surface defining a chamber, an open proximal end, a distal end having a needle cannula attached thereto, said needle cannula having a lumen therethrough in fluid communication with said chamber, said needle cannula projecting distally outwardly from said distal end of said barrel and terminating in a distal tip;
a safety shield mounted on said barrel for movement relative to said barrel from a retracted position in which said safety shield does not materially obstruct access to said distal tip of said needle cannula and an extended position in which said safety shield obstructs access to said distal tip;
means for repeatedly latching and unlatching said safety shield in said extended position;
whereby said safety shield may be conveniently and repeatedly engaged and disengaged by a healthcare worker in order to prevent or allow access to said distal tip of said needle cannula as required by a given medical procedure.

2. The safety needle device of claim 1 wherein said means for repeatedly latching and unlatching said safety shield is comprised of a manually depressible actuator which advances a latch biased to retract or extend said safety shield in response to successive depressions of said actuator.

3. The safety needle device of claim 1 further including an actuator connected to said safety shield by a rotary transmission means.

4. The safety needle device of claim 3 wherein said rotary transmission means is a gear, said barrel and said safety shield have rack teeth which mesh with said gear, said gear rotatably mounted to said actuator whereby movement of said actuator results in movement of said safety shield a distance which is greater than movement of said actuator.

5. The safety needle device of claim 1 wherein said barrel is a syringe barrel having a plunger slidably positioned in fluid-tight engagement inside said barrel, said plunger capable of moving fluid from said chamber through said lumen upon its movement toward said distal end, said plunger capable of facilitating the drawing of fluid into said chamber through said lumen upon its movement away from said distal end.

6. The safety needle device of claim 1 wherein said barrel has a gripping surface which conforms to the contours of a users' fingers.

7. The safety needle device of claim 6 wherein said gripping surface align a users' thumb with an actuator.

8. The safety needle device of claim 1 wherein said needle cannula is removably attached to said barrel.

9. The safety needle device of claim 1 further including means for permanently locking said safety shield in said extended position.

10. A method for alternately and repeatedly obstructing and unobstructing access to
a distal tip of a needle cannula comprising:
mounting a needle cannula projecting distally outward from a distal end of a barrel having an inside surface defining a chamber and an open proximal end, said needle cannula having a lumen therethrough in fluid communication with said chamber, said needle cannula terminating in a distal tip;
attaching a safety shield on said barrel for movement relative to said barrel from a retracted position in which said safety shield does not materially obstruct access to said distal tip of said needle cannula and an extended in which said safety shield obstructs access to said distal tip;
providing a latch means which is able to repeatedly latch and unlatch said safety shield in said extended position on said barrel;
whereby said safety shield may be conveniently and repeatedly engaged and disengaged by a healthcare worker in order to prevent or allow access to said distal tip of said needle cannula as required by a given medical procedure.

11. The method of claim 10 further including a gear and an actuator, said barrel and said safety shield having rack teeth, said gear rotatably mounted to said actuator, said gear meshing with said rack teeth of said barrel and said safety shield whereby movement of said actuator results in movement of said safety shield a distance which is greater than movement of said actuator.

12. The method of claim 10 further including ejecting a shipping safety cap, said shipping safety cap having ejection tabs which contact said safety shield.

13. The method of claim 12 wherein ejecting said shipping safety cap includes:
moving said safety shield in the distal direction; and
ejecting said shipping safety as said safety shield moves in the distal direction.

14. A safety needle device comprising:
a barrel having an inside surface defining a chamber, an open proximal end, a distal end having a needle cannula attached thereto, said needle cannula having a lumen therethrough in fluid communication with said chamber, said needle cannula projecting distally outwardly from said distal end of said barrel and terminating in a distal tip;
a safety shield mounted on said barrel for movement relative to said barrel from a retracted position in which said safety shield does not materially obstruct access to said distal tip of said needle cannula and an extended position in which said safety shield obstructs access to said distal tip;
an actuator connected to said safety shield by a rotary transmission means in order to move said safety shield a multiple of the distance moved by said actuator.
Whereby said safety shield may be moved into the extended position with a relatively short motion of said actuator.

15. The safety needle device of claim 14 wherein said rotary transmission means is a gear, said barrel and said safety shield have rack teeth which mesh with said gear, said gear rotatably mounted to said actuator whereby movement of said actuator results in movement of said safety shield a distance which is greater than movement of said actuator.
